SEQUENCE LISTING (2 sequences)

SEQ ID NO: 1 - DNA, 30 bp, Artificial Sequence
  Primer
agcgttttagcgccggcgccggtgcaggac

SEQ ID NO: 2 - DNA, 38 bp, Artificial Sequence
  Primer
cgccggcgctaaaacgctttcctggaggagctgcggcc

(19) United States
(12) Patent Application Publication
(10) Pub. No.: US 2009/0075331 A1
(43) Pub. Date: Mar. 19, 2009

(54) Industrial-scale serum-free production of recombinant proteins in mammalian cells

(75) Inventor: Ida Molgaard Knudsen, Vaerlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/713,398

(22) Filed: Mar. 2, 2007

Related U.S. Application Data
(63) Continuation of application No. 10/394,085, filed on Mar. 21, 2003.
Application No. 10/394,085 is a continuation of application No. PCT/DK01/00632, filed on Oct. 2, 2001.
(60) Provisional application No. 60/276,322, filed on Mar. 16, 2001.
(60) Provisional application No. 60/271,581, filed on Feb. 26, 2001.
(60) Provisional application No. 60/238,944, filed on Oct. 10, 2000.

(30) Foreign Application Priority Data
Oct. 2, 2000 (DK) PA 2000 01456
Feb. 16, 2001 (DK) PA 2001-00262
Mar. 14, 2001 (DK) PA 2001-00430
May 14, 2001 (DK) PA 2001 00751

(51) Int. Cl. C12P 21/06; C12P 21/04; C12N 5/02

(52) U.S. Cl. 435/69.1; 435/70.3; 435/383; 435/358

(57) ABSTRACT

The invention relates to methods for cultivating mammalian cells and for producing recombinant proteins in large-scale cultures of such cells. The proteins are, e.g., Factor VII or Factor VII-related polypeptides.

### CROSS-REFERENCE TO RELATED APPLICATIONS

This application claims priority under 35 U.S.C. 119 of Danish application no. PA 2000 01456 filed on Oct. 2, 2000; Danish application no. PA 2001 00262 filed on Feb. 16, 2001; Danish application no. PA 2001 00430 filed on Mar. 14, 2001; Danish application no. PA 2001 00751 filed on May 14, 2001; U.S. application No. 60/238,944 filed on Oct. 10, 2000; U.S. provisional application No. 60/271,581 filed on Feb. 26, 2001 and U.S. provisional application No. 60/276,322 filed on Mar. 16, 2001, and claims priority under 35 U.S.C. 120 of international application no. PCT/DK01/00632 filed Oct. 2, 2001, the contents of which are fully incorporated herein by reference.

### FIELD OF THE INVENTION

The present invention relates to methods for cultivating mammalian cells and for producing recombinant proteins in large-scale cultures of such cells.

### BACKGROUND OF THE INVENTION

The proteins involved in the clotting cascade, including, e.g., Factor VII, Factor VIII, Factor IX, Factor X, and Protein C, are proving to be useful therapeutic agents to treat a variety of pathological conditions. Because of the many disadvantages of using human plasma as a source of pharmaceutical products, it is preferred to produce these proteins in recombinant systems. The clotting proteins, however, are subject to a variety of co- and post-translational modifications, including, e.g., asparagine-linked (N-linked) glycosylation; O-linked glycosylation; and γ-carboxylation of glu residues. For this reason, it is preferable to produce them in mammalian cells, which are able to modify the recombinant proteins appropriately. Mammalian cell culture, however, has traditionally been performed in the presence of animal serum or other animal derived components. Methods for serum-free cultivation have produced variable results. In particular, cultivation of cells in the absence of serum from initiation of the culture until attainment of large-scale production volumes has been problematic. Anchorage-dependent cell lines are usually grown in serum-containing medium, which may then be exchanged for serum-free medium for a particular purpose, such as, e.g., accumulation of a secreted protein in the culture medium. Large-scale suspension cultures present other difficulties, including, e.g., a lack of reliable devices for retention of suspension cells in the culture vessel.

Thus, there is a need in the art for methods for large-scale mammalian cell culture to produce industrial quantities of clotting proteins, particularly recombinant human Factor VII or Factor VII-related polypeptides.

### SUMMARY OF THE INVENTION

The present invention provides methods for large-scale production of Factor VII or a Factor VII-related polypeptide in mammalian cells, which are carried out by the steps of:

(i) inoculating Factor VII-expressing or Factor VII-related polypeptide-expressing mammalian cells into a seed culture vessel containing medium lacking animal-derived components and propagating the seed culture at least until the cells reach a minimum cross-seeding density;

(ii) transferring the propagated seed culture, or a portion thereof, to a large-scale culture vessel containing (a) medium lacking animal-derived components and (b) macroporous carriers, under conditions in which the cells migrate into the carriers;

(iii) propagating the large-scale culture in medium lacking animal-derived components, at least until the cells reach a predetermined density;

(iv) maintaining the culture obtained in step (iii) in medium lacking animal-derived components, under conditions appropriate for Factor VII expression or Factor VII-related polypeptide expression; and

(v) recovering the Factor VII or the Factor VII-related polypeptide from the maintained culture.

Preferably, the macroporous carriers:
(a) have an overall particle diameter between about 150 and 350 um;
(b) have pores having an average pore opening diameter of between about 15 and about 40 um; and
(c) have a positive charge density of between about 0.8 and 2.0 meq/g.

In some embodiments, the cells have been adapted to grow in medium lacking animal-derived proteins and/or in suspension culture. In some embodiments, the cells used have been adapted to grow in suspension culture in medium lacking animal-derived components prior to inoculation in step (i). Preferably, Factor VII or a Factor VII-related polypeptide is produced at a level at least about 1 mg/l of culture, more preferably at least about 2.5 mg/l of culture, more preferably at least about 5 mg/l of culture and most preferably at least about 8 mg/l of culture.

In another aspect, the present invention provides methods for large-scale cultivation of mammalian cells, which are carried out by the steps of:

(i) inoculating cells into a seed culture vessel containing medium lacking animal-derived components and propagating the seed culture at least until the cells reach a minimum cross-seeding density;

(ii) transferring the propagated seed culture to a large-scale culture vessel containing (a) medium lacking animal-derived components and (b) macroporous carriers, under conditions in which the cells migrate into the carriers, and

(iii) propagating the large-scale culture in medium lacking animal-derived proteins, at least until the cells reach a predetermined density.

In some embodiments, the method further comprises:

(iv) maintaining the culture obtained in step (iii) in medium lacking animal-derived components by regular harvesting of the culture medium and replacement by fresh medium.

In one embodiment thereof, the method comprises:

(iv) maintaining the culture obtained in step (iii) in medium lacking animal-derived components by continuous perfusion, i.e. by continuous harvesting of culture medium, using a retention device to retain the cell-containing carriers in the culture vessel, and continuous addition of fresh medium.

In another embodiment thereof, the method comprises:

(iv) maintaining the culture obtained in step (iii) in medium lacking animal derived components by regular harvesting of part the culture supernatant after sedimentation of the cell-containing carriers and replacement with fresh medium.

In some embodiments, the method further comprises:

(v) cooling the culture to a pre-determined temperature below the temperature setpoint of the cultivation before the sedimentation of carriers (from 5 to 30° C., such as, e.g., from 5 to 20° C., or from 5 to 15° C. or to about 10° C. below setpoint).

In some embodiments, the cells produce a desired polypeptide, preferably a clot-ting factor and most preferably human Factor VII or a human Factor VII-related polypeptide, including, without limitation, wild-type Factor VII, S52A-Factor VII, S60A-Factor VII, R152E-Factor VII, S344A-Factor VII, and Factor VIIa lacking the Gla domain.

### DETAILED DESCRIPTION OF THE INVENTION

The present invention provides methods for large-scale cultivation of mammalian cells, particularly to produce industrial amounts of desired polypeptides that are expressed by such cells. In one aspect, the invention relates to cultivation of suspension-competent mammalian cells in medium lacking animal-derived components. In another aspect, the present invention is based on the discovery that the use of macroporous carriers having a positive surface charge provides a suitable environment for the propagation of suspension-competent cells in the absence of animal-derived components and allows high-level production of desired proteins by such cells.

### Cells:

In practicing the present invention, the cells being cultivated are preferably mammalian cells, more preferably an established mammalian cell line, including, without limitation, CHO (e.g., ATCC CCL 61), COS-1 (e.g., ATCC CRL 1650), baby hamster kidney (BHK), and HEK293 (e.g., ATCC CRL 1573; Graham et al., J. Gen. Virol. 36:59-72,1977) cell lines.

A preferred BHK cell line is the tk⁻ ts13 BHK cell line (Waechter and Baserga, Proc. Natl. Acad. Sci. USA 79:1106-1110, 1982), hereinafter referred to as BHK 570 cells. The BHK 570 cell line is available from the American Type Culture Collection, 12301 Parklawn Dr., Rockville, Md. 20852, under ATCC accession number CRL 10314. A tk⁻ ts13 BHK cell line is also available from the ATCC under accession number CRL 1632.

A preferred CHO cell line is the CHO K1 cell line available from ATCC under accession number CCI61. Other suitable cell lines include, without limitation, Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1); DUKX cells (CHO cell line) (Urlaub and Chasin, Proc. Natl. Acad. Sci. USA 77:4216-4220, 1980) (DUKX cells also being referred to as DXB11 cells), and DG44 (CHO cell line) (Cell, 33: 405, 1983, and Somatic Cell and Molecular Genetics 12: 555, 1986). Also useful are 3T3 cells, Namalwa cells, myelomas and fusions of myelomas with other cells. In some embodiments, the cells may be mutant or recombinant cells, such as, e.g., cells that express a qualitatively or quantitatively different spectrum of enzymes that catalyze post-translational modification of proteins (e.g., glycosylation enzymes such as glycosyl transferases and/or glycosidases, or processing enzymes, or processing or stabilizing proteins, such as, for example, propeptides) than the cell type from which they were derived.

In some embodiments, the cells used in practicing the invention are capable of growing in suspension cultures. As used herein, suspension-competent cells are those that can grow in suspension without making large, firm aggregates, i.e., cells that are monodisperse or grow in loose aggregates with only a few cells per aggregate. Suspension-competent cells include, without limitation, cells that grow in suspension without adaptation or manipulation (such as, e.g., hematopoietic cells or lymphoid cells) and cells that have been made suspension-competent by gradual adaptation of attachment-dependent cells (such as, e.g., epithelial or fibroblast cells) to suspension growth.

### Medium:

The present invention encompasses cultivating mammalian cells in medium lacking animal-derived components. As used herein, “animal-derived” components are any components that are produced in an intact animal (such as, e.g., proteins isolated and purified from serum) or are components produced by using components produced in an intact animal (such as, e.g., an amino acid made by using an enzyme isolated and purified from an animal to hydrolyse a plant source material).

By contrast, a protein which has the sequence of an animal protein (i.e., has a genomic origin in an animal) but which is produced in vitro in cell culture (such as, e.g., in a recombinant yeast or bacterial cell or in an established continuous mammalian cell line, recombinant or not), in media lacking components that are isolated and purified from an intact animal is not an “animal-derived” component (such as, e.g., insulin produced in a yeast or a bacterial cell, or insulin produced in an established mammal cell line, such as, e.g., CHO, BHK or HEK cells, or interferon produced in Namalwa cells). For example, a protein which has the sequence of an animal protein (i.e., has a genomic origin in an animal) but which is produced in a recombinant non-animal cell (such as, e.g., insulin produced in a yeast or bacterial cell) is not an “animal-derived” component. Accordingly, a cell culture medium lacking animal-derived components is one that may contain animal proteins that are recombinantly produced; such medium, however, does not contain, e.g., animal serum or proteins or other products purified from animal serum. Further-more, the medium does not contain any other components, such as, e.g., lipids, or amino acids isolated and purified from an intact animal. Such medium may, for example, contain one or more components derived from plants.

Any cell culture medium lacking animal-derived components that supports cell growth and maintenance under the conditions of the invention may be used. Typically, the medium contains water, an osmolality regulator, a buffer, an energy source, amino acids, an inorganic or recombinant iron source, one or more synthetic or recombinant growth factors, vitamins, and cofactors. In addition to conventional components, a medium suitable for producing Factor VII contains Vitamin K1, which facilitates γ-carboxylation of glutamic acid residues in Factor VII, at a concentration between about 0.1-50 mg/liter, preferably between about 0.5-25 mg/liter, more preferably between about 1-10 mg/liter and most preferably about 5 mg/liter.

In one embodiment, the medium used has the following composition:

| COMPONENT | |
|---|---|
| | MG/L |
| Sodium Chloride | 6122 |
| Potassium Chloride | 311.8 |
| Sodium Dihydrogen Phosphate Monohydrate | 62.5 |
| Disodium Hydrogen Phosphate Anhydrous | 71.02 |
| Magnesium Chloride Anhydrous | 28.64 |
| Magnesium Sulphate Anhydrous | 48.84 |
| Calcium Chloride Anhydrous | 116.6 |
| Copper Sulphate 5-hydrate | 0.0013 |
| Ferrous Sulphate 7-hydrate | 0.417 |
| Ferric Nitrate 9-hydrate | 0.05 |
| Zinc Sulphate 7-hydrate | 0.432 |
| Dextrose Anhydrous | 4501 |
| Linoleic Acid | 1.189 |
| DL-68-Thioctic Acid | 0.473 |
| L-Alanine | 4.45 |
| L-Arginine Hydrochloride | 547.5 |
| L-Asparagine Monohydrate | 407.5 |
| L-Aspartic Acid | 6.65 |
| L-Cysteine Hydrochloride Monohydrate | 117.65 |
| L-Glutamic Acid | 251.35 |
| L-Glutamine | 365 |
| Glycine | 18.75 |
| L-Histidine Hydrochloride Monohydrate | 211.48 |
| L-Isoleucine | 54.47 |
| L-Leucine | 179.05 |
| L-Lysine Hydrochloride | 231.25 |
| L-Methionine | 137.24 |
| L-Phenylalanine | 155.48 |
| L-Proline | 17.25 |
| L-Serine | 266.25 |
| L-Threonine | 173.45 |
| L-Tryptophan | 39.02 |
| L-Tyrosine Disodium Dihydrate | 55.79 |
| L-Valine | 177.85 |
| L-Cystine Dihydrochloride | 31.29 |
| Sodium Hypoxanthine | 2.39 |
| Putrescine Dihydrochloride | 0.081 |
| Sodium Pyruvate | 220 |
| D-Biotin | 0.1313 |
| D-Calcium Pantothenate | 4.08 |
| Folic Acid | 4.65 |
| I-Inositol | 39.1 |
| Nicotinamide | 3.085 |
| Choline Chloride | 29.32 |
| Pyridoxine Hydrochloride | 0.117 |
| Riboflavin | 0.219 |
| Thiamine Hydrochloride | 2.67 |
| Thymidine | 0.365 |
| Vitamin B12 | 2.68 |
| Pyridoxal Hydrochloride | 3 |
| Glutathione | 2.5 |
| Sodium Selenite | 0.02175 |
| L-Ascorbic Acid, Free Acid | 27.5 |
| Sodium Hydrogen Carbonate | 2440 |
| HySoy (soy protein hydrolysate) | 500 |
| Ethanolamin | 1.22 |
| Insulin | 5 |
| Dextran T70 | 100 |
| Pluronic F68 | 1000 |
| Vitamin K1 | 5 |
| | ML/L |
| Fe/citrat complex (50 mM/1 M) | 0.4 |
| Mercaptoethanol | 0.0035 |

In preferred embodiments, the cells used in practicing the present invention are adapted to suspension growth in medium lacking animal-derived components, such as, e.g., medium lacking serum, or medium lacking animal-derived components and proteins. Such adaptation procedures are described, e.g., in Scharfenberg, et al., Animal Cell Technology Developments towards the 21^{st} Century, E. C. Beuvery et al. (Eds.), Kluwer Academic Publishers, pp. 619-623, 1995 (BHK and CHO cells); Cruz, et al., Biotechnol. Tech. 11:117-120,1997 (insect cells); Keen & Steward, Cytotechnol. 17:203-211, 1995 (myeloma cells); Berg et al., Biotechniques 14:972-978, 1993 (human kidney 293 cells).

In a particularly preferred embodiment, the host cells are BHK 21 or CHO cells that have been engineered to express human Factor VII or a Factor VII-related polypeptide, and that have been adapted to grow in the absence of serum or animal-derived components.

### Culture Methods

The present invention provides methods for large-scale cultivation of mammalian cells, which are carried out by the steps of:

(i) inoculating cells into a seed culture vessel containing culture medium lacking animal-derived components and propagating the seed culture at least until the cells reach a minimum cross-seeding density;

(ii) transferring the propagated seed culture to a large-scale culture vessel containing (a) culture medium lacking animal-derived components and (b) macroporous carriers, under conditions in which the cells migrate into the carriers; and

(iii) propagating the large-scale culture in medium lacking animal-derived components, at least until said cells reach a useful density.

In some embodiments, the methods further comprise the step of:

(iv) maintaining the culture obtained in step (iii) in medium lacking animal-derived components by regular harvesting of the culture medium and replacement by fresh medium.

In some embodiments thereof, the methods comprise:

(iv) maintaining the culture obtained in step (iii) in medium lacking animal-derived components by continuous perfusion, i.e. by continuous harvesting of culture medium, using some sort of retention device to retain the cell-containing carriers in the culture vessel, and continuous addition of fresh medium;

In some embodiments thereof, the methods comprise:

(iv) maintaining the culture obtained in step (iii) in medium lacking animal derived components by regular harvesting of part the culture supernatant after sedimentation of the cell-containing carriers and replacement with fresh medium.

In some embodiments, the method further comprises:

(v) cooling the culture to a pre-determined temperature below the temperature setpoint of the cultivation before each sedimentation of carriers.

In further embodiments, the temperature is lowered from 5 to 30° C., or from 5 to 20° C., or from 5 to 15° C., or to about 10° C. below the setpoint of the cultivation.

Inoculation and initial propagation: It will be understood that step (i) may be repeated with a progressive increase in the size of the seed culture vessel, until a sufficient number of cells is obtained for step (ii). For example, one or more seed culture vessels of 5 l, 50 l, or 500 l may be used sequentially. A seed culture vessel as used herein is one that has a capacity of between about 5 l and 500 l. Typically, cells are inoculated into a seed culture vessel at an initial density of about 0.2-0.4×10⁶ cells/ml and propagated until the culture reaches a cell density of about 1.0×10⁶ cells/ml. As used herein, a minimum cross-seeding density is between about 0.8 and about 1.5×10⁶ cells/ml.

Macroporous carriers: As used herein, macroporous carriers are particles, usually cellulose-based, which have the following properties: (a) They are small enough to allow them to be used in suspension cultures (with a stirring rate that does not cause significant shear damage to cells); (b) They have pores and interior spaces of sufficient size to allow cells to migrate into the interior spaces of the particle and (c) Their surfaces (exterior and interior) are positively charged. In one series of embodiments, the carriers:

(a) have an overall particle diameter between about 150 and 350 um;

(b) have pores having an average pore opening diameter of between about 15 and about 40 um; and

(c) have a positive charge density of between about 0.8 and 2.0 meq/g. In some embodiments, the positive charge is provided by DEAE (N, N,-diethylaminoethyl) groups. Useful macroporous carriers include, without limitation, Cytopore 1™ and Cytopore 2™ (Amersham Pharmacia Biotech, Piscataway N.J.). Particularly preferred are Cytopore 1™ carriers, which have a mean particle diameter of 230 um, an average pore size of 30 um, and a positive charge density of 1.1 meq/g.

Large-scale culture conditions: As used herein, a large-scale culture vessel has a capacity of at least about 100 l, preferably at least about 500 l, more preferably at least about 1000 l and most preferably at least about 5000 l. Typically, step (ii) involves transferring about 50 l of the propagated seed culture (having about 1.0×10⁶ cells/ml) into a 500 l culture vessel containing 150 l of culture medium and 750 g macroporous carriers.

After the transfer, the cells typically migrate into the interior of the carriers within the first 24 hours. The large-scale culture is maintained under appropriate conditions of, e.g., temperature, pH, dissolved oxygen tension (DOT), and agitation rate, and the volume is gradually increased by adding medium to the culture vessel.

High-level protein expression: When the cells are being propagated in order to produce high levels of a desired protein, steps (i), (ii), and (iii) are designated the “growth” phase and step (iv) is designated the “production” phase. In the production phase, the medium is typically exchanged at 24-h intervals by sedimentation of the cell-containing carriers; harvesting of the culture supernatant; and replacement with fresh medium. A cooling step may be applied immediately before the sedimentation of carriers (cooling down to from 5 to 30° C., such as, e.g. from 5 to 20° C., or from 5 to 15° C., or about 10° C. below the temperature set point of the cultivation) to reduce the oxygen requirement of the cells while sedimented at the bottom of the culture vessel The cooling step is done over 10-240 minutes, such as, e.g., 20-180 minutes, or 30-120 minutes before sedimenting the cell-containing macropororous carriers. The step is typically carried out as follows: The bioreactor is cooled and the temperature is monitored. When the bioreactor reaches a pre-determined temperature below the setpoint of the cultivation, such as, e.g., 10° C. below the setpoint, the agitator of the bioreactor is stopped and the cell-containing carriers are sedimented. When media exchange has taken place, the temperature is again regulated to the setpoint of the cultivation. The fresh media being added is typically pre-warmed to a temperature close to the setpoint of the cultivation.

Alternatively, a continuous perfusion mode of culture may be used in which culture medium is continuously harvested, using some sort of retention device, e.g., some sort of settling device, to retain the carriers in the culture vessel, and fresh medium is continuously added.

Once the medium has been removed from the culture vessel, it may be subjected to one or more processing steps to obtain the desired protein, including, without limitation, centrifugation or filtration to remove cells that were not immobilized in the carriers; affinity chromatography, hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J.-C. Janson and Lars Ryden, editors, VCH Publishers, New York, 1989.

Purification of Factor VII or Factor VII-related polypeptides may involve, e.g., affinity chromatography on an anti-Factor VII antibody column (see, e.g., Wakabayashi et al., J. Biol. Chem. 261:11097, 1986; and Thim et al., Biochem. 27:7785, 1988) and activation by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat. No. 4,456,591; and Hedner et al., J. Clin. Invest. 71:1836 (1983). Alternatively, Factor VII may be activated by passing it through an ion-exchange chromatography column, such as Mono Q® (Pharmacia) or the like.

### Polypeptides for Large-Scale Production

In some embodiments, the cells used in practicing the invention are human cells expressing an endogenous Factor VII gene. In these cells, the endogenous gene may be intact or may have been modified in situ, or a sequence outside the Factor VII gene may have been modified in situ to alter the expression of the endogenous Factor VII gene.

In other embodiments, cells from any mammalian source are engineered to express human Factor VII from a recombinant gene. As used herein, “Factor VII” or “Factor VII polypeptide” encompasses wild-type Factor VII (i.e., a polypeptide having the amino acid sequence disclosed in U.S. Pat. No. 4,784,950), as well as variants of Factor VII exhibiting substantially the same or improved biological activity relative to wild-type Factor VII. The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa.

As used herein, “Factor VII-related polypeptides” encompasses polypeptides, including variants, in which the Factor VIIa biological activity has been substantially modified or reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce of Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) and (iv) measuring hydrolysis of a synthetic substrate.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids. Non-limiting examples of Factor VII variants having substantially the same biological activity as wild-type Factor VII include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); and oxidized forms of Factor VIIa (Kornfelt et al., Arch. Biochem. Biophys. 363:43-54, 1999). Non-limiting examples of Factor VII variants having substantially reduced or modified biological activity relative to wild-type Factor VII include R152E-FVIIa (Wildgoose et al., Biochem 29:3413-3420, 1990), S344A-FVIIa (Kazama et al., J. Biol. Chem. 270:66-72, 1995), FFR-FVIIa (Holst et al., Eur. J. Vasc. Endovasc. Surg. 15:515-520, 1998), and Factor VIIa lacking the Gla domain, (Nicolaisen et al., FEBS Letts. 317:245-249, 1993).

The present invention also encompasses large-scale cultivation of mammalian cells that express one or more proteins of interest, whether from endogenous genes or subsequent to introduction into such cells of recombinant genes encoding the protein. Such proteins include, without limitation, Factor VIII; Factor IX; Factor X; Protein C; tissue factor; rennin; growth hormone, including human growth hormone; bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β platelet-derived growth factor (PDGF); fibroblast growth factor such as α-FGF and β-FGF; epidermal growth factor (EGF); transforming growth factor (TGF) such as TGF-alpha and TGF-beta, insulin-like growth factor-I and -II (IGF-I and IGF-II); CD proteins such as CD-3, CD-4, CD-8, and CD-19; erythropoietin; osteoinductive factors; immunotoxins; bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors (jeg kender ikke udtrykket homing receptors, men g\dot{a}r ud fra at du ved det er OK?, IMq, 2001-09-18); addressin; regulatory proteins; antibodies; and fragments of any of the above polypeptides.

The following examples are intended as non-limiting illustrations of the present invention.

### EXAMPLE 1

### Serum-free Production of Factor VII

The following experiment was performed to produce Factor VII in large-scale culture.

A BHK cell line transformed with a Factor VII-encoding plasmid was adapted to growth in suspension culture in the absence of serum. After adaptation the cells were propagated sequentially in spinner cultures; as the cell number increased, the volume was gradually increased by addition of new medium. The medium used was free of serum and other animal derived components.

Finally, 6 l of seed culture were inoculated into a 100-liter production bioreactor containing macroporous Cytopore 1 carriers (Amersham Pharmacia Biotech), after which the suspension cells became immobilized in the carriers within 24 hours after inoculation. The culture was maintained at 36° C. at a pH of 6.7-6.9 and a dissolved oxygen tension (DOT) of 50% of saturation. The volume in the production bioreactor was gradually increased by addition of new medium as the cell number increased. When the cell density reached approximately 2×10⁶ cells/ml, the production phase was initiated and a medium change was performed every 24 hours: Agitation was stopped to allow for sedimentation of the cell-containing carriers, and 80% of the culture supernatant was then harvested and replaced with new medium. The harvested culture supernatant was filtered to remove non-trapped cells (i.e. cells that had not been immobilised in the carriers) and cell debris and was then transferred for further processing.

During the production phase the cells reached 3-6×10⁶ cells/ml and a titer of 2-7 mg Factor VII/liter.

### EXAMPLE 2

### Serum Free Production of Factor VII

The following experiment was performed to produce Factor VII in large-scale culture.

A plasmid vector pLN174 for expression of human FVII has been described (Persson and Nielsen. 1996. FEBS Lett. 385: 241-243). Briefly, it carries the cDNA nucleotide sequence encoding human FVII including the propeptide under the control of a mouse metallothionein promoter for transcription of the inserted cDNA, and mouse dihydrofolate reductase cDNA under the control of an SV40 early promoter for use as a selectable marker.

For construction of a plasmid vector encoding a gamma-carboxylation recognition sequence, a cloning vector pBluescript II KS+ (Stratagene) containing cDNA encoding FVII including its propeptide was used (pLN171). (Persson et al. 1997. J. Biol. Chem. 272: 19919-19924). A nucleotide sequence encoding a stop codon was inserted into the cDNA encoding FVII after the propeptide of FVII by inverse PCR-mediated mutagenesis on this cloning vector. The template plasmid was denatured by treatment with NaOH followed by PCR with Pwo (Boehringer-Mannheim) and Taq (Perkin-Elmer) polymerases with the following primers:

a) 5′-AGC GTT TTA GCG CCG GCG CCG GTG CAG GAC-3′
b) 5′-CGC CGG CGC TAA AAC GCT TTC CTG GAG GAG CTG CGG CC-3′

The resulting mix was digested with DpnI to digest residual template DNA and Escherichia coli were transformed with the PCR product. Clones were screened for the presence of the mutation by sequencing. The cDNA from a correct clone was transferred as a BamHI-EcoRI fragment to the expression plasmid pcDNA3 (Invitrogen). The resulting plasmid was termed pLN329. CHO K1 cells (ATCC CCI61) were transfected with equal amounts of pLN174 and pLN329 with the Fugene6 method (Boehringer-Mannheim). Transfectants were selected by the addition of methotrexate to 1 μM and G-418 to 0.45 mg/ml. The pool of transfectants were cloned by limiting dilution and FVII expression from the clones was measured.

A high producing clone was further subcloned and a clone E11 with a specific FVII expression of 2.4 pg/cell/day in Dulbecco-modified Eagle's medium with 10% fetal calf serum was selected. The clone was adapted to serum free suspension culture in a commercially available CHO medium free of animal-derived components.

The adapted cells were propagated sequentially in spinner cultures and as the cell number increased, the volume was gradually increased by addition of new medium.

After 25 days, 6 l of spinner culture were inoculated into a 50-liter bioreactor. The cells were propagated in the bioreactor and as the cell number increased, the volume was gradually increased by addition of new medium.

Finally, 50 l of seed culture were inoculated into a 500-liter production bioreactor containing macroporous Cytopore 1 carriers (Amersham Pharmacia Biotech), after which the suspension cells became immobilized in the carriers. The culture was maintained at 36° C. at a pH of 7.0-7.1 and a Dissolved Oxygen Tension (DOT) of 50% of saturation. The volume in the bioreactor was gradually increased by addition of new medium as the cell number increased. When the cell density reached approximately 10-12×105 cells/ml, the production phase was initiated and a medium change was performed every 24 hours: agitation was stopped to allow for sedimentation of the cell-containing carriers, and 80% of the culture supernatant was then harvested and replaced with new medium. The harvested culture supernatant was filtered to remove non-trapped cells (i.e. cells that were not immobilized in carriers) and cell debris and was then transferred for further processing.

During the production phase the cells reached 2-3×10⁷ cells/ml and a titer of 8 mg factor VII/liter.

### EXAMPLE 3

### Serum Free Production of Factor VII

The following experiment was performed to produce Factor VII in large-scale culture.

A high producing CHO clone was made as described in Example 2.

The medium used was free of animal derived components.

The adapted cells were propagated sequentially in spinner cultures and as the cell number increased, the volume was gradually increased by addition of new medium.

After 25 days, 6 l of spinner culture were inoculated into a 50-liter bioreactor. The cells were propagated in the bioreactor and as the cell number increased, the volume was gradually increased by addition of new medium.

Finally, 50 l of seed culture were inoculated into a 500-liter production bioreactor containing macroporous Cytopore 1 carriers (Amersham Pharmacia Biotech), after which the suspension cells became immobilized in the carriers. The culture was maintained at 36° C. at a pH of 7.0-7.1 and a Dissolved Oxygen Tension (DOT) of 50% of saturation. The volume in the bioreactor was gradually increased by addition of new medium as the cell number increased. When the cell density reached approximately 10-12×105 cells/ml, the production phase was initiated and a medium change was performed every 24 hours: agitation was stopped to allow for sedimentation of the cell-containing carriers, and 80% of the culture supernatant was then harvested and replaced with new medium. The harvested culture supernatant was filtered to remove non-trapped cells (i.e. cells that were not immobilized in carriers) and cell debris and was then transferred for further processing.

From day 14 onwards the medium was fortified with 2 g/l of HY—SOY (hydrolyzed soy protein).

From day 41 onwards cooling of the culture to 10° C. below setpoint (i.e. to 26° C.) immediately before the daily medium exchange was introduced. The idea of the cooling step was to reduce the oxygen requirements of the cells before the agitation was stopped and the carriers with cells were left to sediment at the bottom of the fermentor.

During the production phase the cells reached 2.5-3.5×10⁷ cells/ml and a titer of 8-13 mg factor VII/liter.

All patents, patent applications, and literature references referred to herein are hereby incorporated by reference in their entirety.

Many variations of the present invention will suggest themselves to those skilled in the art in light of the above detailed description. Such obvious variations are within the full intended scope of the appended claims.

1. A method for large-scale production of Factor VII in mammalian cells, said method comprising:
(i) inoculating Factor VII-expressing or mammalian cells that have been adapted to grow in suspension culture in the absence of animal-derived components into a seed culture vessel containing medium lacking animal-derived components and propagating said seed culture in suspension at least until the cells reach a minimum cross-seeding density;
(ii) transferring said propagated seed culture to a culture vessel having a capacity of at least about 100 liters containing (a) medium lacking animal-derived components and (b) macroporous carriers, under conditions in which said cells migrate into the carriers;
(iii) propagating said culture in medium lacking animal-derived components, at least until said cells reach a predetermined density;
(iv) maintaining the culture obtained in step (iii) in medium lacking animal-derived components, under conditions appropriate for Factor VII expression; and
(v) recovering the Factor VII from the maintained culture.

2. A method as defined in claim 1, wherein said macroporous carriers:
(a) have an overall particle diameter between about 150 and 350 um;
(b) have pores having an average pore opening diameter of between about 15 and about 40 um; and
(c) have a positive charge density of between about 0.8 and 2.0 meq/g.

3-4. (canceled)

5. A method as defined in claim 1, wherein Factor VII is produced at a level at least about 1 mg/l of culture.

6. A method as defined in claim 5, wherein Factor VII is produced at a level at least about 2.5 mg/l of culture.

7. A method as defined in claim 6, wherein Factor VII is produced at a level at least about 5 mg/l of culture.

8. A method as defined in claim 7, wherein Factor VII is produced at a level at least about 8 mg/l of culture.

9. A method for large-scale cultivation of mammalian cells, said method comprising:
(i) inoculating cells that have been adapted to grow in suspension culture in the absence of animal-derived components into a seed culture vessel containing medium lacking animal-derived components and propagating said seed culture in suspension at least until the cells reach a minimum cross-seeding density;
(ii) transferring said propagated seed culture to a culture vessel having a capacity of at least about 100 liters containing (a) medium lacking animal-derived components and (b) macroporous carriers, under conditions in which said cells migrate into the carriers, and
(iii) propagating said culture in medium lacking animal-derived components, at least until said cells reach a predetermined density.

10. A method as defined in claim 9, further comprising:
(iv) maintaining the culture obtained in step (iii) in medium lacking animal derived components by regular harvesting of the culture medium and replacement by fresh medium.

11. A method as defined in claim 10, step (iv) comprising:
(iv) maintaining the culture obtained in step (iii) in medium lacking animal derived components by continuous perfusion, i.e. by continuous harvesting of culture medium, using a retention device to retain the cell-containing carriers in the culture vessel, and continuous addition of fresh medium;

12. A method as defined in claim 10, step (iv) comprising:
(iv) maintaining the culture obtained in step (iii) in medium lacking animal derived components by regular harvesting of part the culture supernatant after sedimentation of the cell-containing carriers and replacement with fresh medium.

13. A method as defined in claim 12, further comprising:
(v) cooling of the culture to a pre-determined temperature below the temperature setpoint of the cultivation before the sedimentation of carriers.

14. A method as defined in claim 13, where the culture is cooled to a temperature of from 5° C. to 30° C. below the temperature setpoint of the cultivation before the sedimentation of carriers.

15. A method as defined in claim 14, where the culture is cooled to a temperature of from 5° C. to 20° C. below the temperature setpoint of the cultivation.

16. A method as defined in claim 15, where the culture is cooled to a temperature of from 5° C. to 15° C. below the temperature setpoint of the cultivation.

17. A method as defined in claim 16, where the culture is cooled to a temperature of about 10° C. below the temperature setpoint of the cultivation.

18. A method as defined in claim 9, wherein said macroporous carriers:
(a) have an overall particle diameter between about 150 and 350 um;
(b) have pores having an average pore opening diameter of between about 15 and about 40 um; and
(c) have a positive charge density of between about 0.8 and 2.0 meq/g.

19-20. (canceled)

21. A method as defined in claim 9, wherein said cells produce a desired polypeptide.

22. A method as defined in claim 21, wherein said desired polypeptide is human Factor VII.

23. A method as defined in claim 21, wherein the desired polypeptide is selected from the group consisting of: wild-type Factor VII, S52A-Factor VII, S60A-Factor VII.

24. A method as defined in claim 9, wherein the mammalian cell is selected from the group consisting of BHK cells and CHO cells.

25. A method as defined in claim 9, wherein said macroporous carriers are cellulose-based.

26. A method as defined in claim 9, wherein said macroporous carriers comprise surface DEAE groups that impart said charge density.

27. A method as defined in claim 9, further comprising, prior to step (ii), repeating step (i) using seed culture vessels of progressively increasing size.

28. A method for producing a polypeptide, said method comprising:
(i) providing a mammalian cell expressing said polypeptide, wherein said cell has been adapted to grow in suspension culture in the absence of animal-derived components;
(ii) inoculating said cell into a seed culture vessel containing medium lacking animal-derived components and propagating said seed culture in suspension at least until the cells reach a minimum cross-seeding density;
(iii) transferring said propagated seed culture to a culture vessel having a capacity of at least about 100 liters containing (a) medium lacking animal-derived components and (b) macroporous carriers, under conditions in which said cells migrate into the carriers, wherein said carriers:
(a) have an overall particle diameter between about 150 and 350 um;
(b) have pores having an average pore opening diameter of between about 15 and about 40 um;
(c) have a positive charge density of between about 0.8 and 2.0 meq/g; and
(iv) propagating said culture in medium lacking animal-derived components, at least until said cells reach a minimum desired density; and
(v) maintaining said culture under conditions in which said polypeptide is produced by said culture.

29. A method as defined in claim 28, wherein said polypeptide is human Fact or VII.

30. A method as defined in claim 28, wherein said cell is selected from the group consisting of BHK cells and CHO cells and wherein said cell is transfected with a human Factor VII-encoding nucleic acid.
